(19) United States
(12) Patent Application Publication
(10) Pub. No.: US 2008/0095834 A1
(43) Pub. Date: Apr. 24, 2008

(54) Mitochondriotropic Phospholipid Vesicles

(76) Inventors: Volkmar Weissig, Nashua, NH (US); Sarathi V. Boddapati, Boston, MA (US); Robert N. Hanson, Newton, MA (US); Vladimir P. Torchilin, Charlestown, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/885,419

(22) PCT Filed: Mar. 2, 2006
(86) PCT No.: PCT/US06/07628

§ 371 Date: Aug. 30, 2007

Related U.S. Application Data
(60) Provisional application No. 60/657,802, filed on Mar. 2, 2005.

(51) Int. Cl. A61K 9/127; A61K 31/70; A61P 43/00; A61K 38/00

(52) U.S. Cl. 424/450; 514/2; 514/44

(57) ABSTRACT

Mitochondriotropic phospholipid vesicles, i.e., mitochondriotropic liposomes, that comprise a hydrophobized amphiphilic delocalized cation, such as those comprising, e.g., a triphenylphosphonium or a quinolinium moiety, incorporated into the phospholipid membrane of the vesicles, or liposomes, are disclosed. The hydrophobized portion of the amphiphilic delocalized cation, e.g., a fatty acid or other phospholipid derivative, is embedded in the phospholipid membrane of the liposome, and the amphiphilic portion of the cation is exposed on the surface of the liposome. Mitochondriotropic liposomes constitute a mitochondria-targeted drug delivery system, permitting the transport of a high payload of therapeutic water-soluble molecules in their native (i.e., active) state specifically and exclusively to mitochondria in living mammalian cells.

### CROSS REFERENCE TO RELATED APPLICATIONS

This application claims the priority of U.S. Provisional Application No. 60/657,802 filed Mar. 2, 2005 entitled, MITOCHONDRIOTROPIC PHOSPHOLIPID VESICLES, the whole of which is hereby incorporated by reference herein.

### STATEMENT REGARDING FEDERALLY SPONSORED RESEARCH OR DEVELOPMENT

N/A

### BACKGROUND OF THE INVENTION

Since the first demonstration in 1988 that mitochondrial DNA (mtDNA) base substitution and deletion mutations are linked to human disease, a variety of degenerative diseases have been associated with mtDNA mutations (Wallace, 1994). Mitochondria are vital for the cell's energy metabolism and for the regulation of programmed cell death. In addition, mitochondria are critically involved in the modulation of intracellular calcium concentration and the mitochondrial respiratory chain. Consequently mitochondrial dysfunction either causes or at least contributes to a large number of human diseases.

Mitochondria are a prime target for pharmacological intervention (Szewczyk et al., 2002). Malfunctioning mitochondria are found in several adult-onset diseases including diabetes, cardiomyopathy, infertility, migraine, blindness, deafness, kidney and liver diseases and stroke. The accumulation of somatic mutations in the mitochondrial genome has been suggested as contributing to aging, age-related neurodegenerative diseases and neuromuscular diseases as well as in cancer. Certain deleterious base substitutions can cause familial deafness and some cases of Alzheimer's disease and Parkinson's disease. Other nucleotide substitutions have been associated with Leber's Hereditary Optic Neuropathy (LHON) and myoclonic epilepsy and ragged-red fiber disease (MERF). Base substitutions can also cause pediatric diseases such as Leigh's syndrome and dystonia. Severe rearrangements involving deletions have been linked with adult-onset chronic progressive external opthalmoplegia (CPEO) and Kearns-Sayre syndrome (KSS) as well as the lethal childhood disorder Pearson's marrow/pancreas syndrome (Wallace, 1994).

Three different approaches for somatic gene therapy (Ledley, 1996) can be distinguished based on the nature of the material that is administered to the patient: (a) cell-based approaches involving the administration to the patient of genetically engineered cells (“ex-vivo”), (b) administration to the patient of genetically engineered, attenuated, or defective viruses, and (c) plasmid-based approaches that involve pharmaceutical formulations of DNA molecules. A variety of viral and non-viral methods have been developed for introducing DNA molecules into a cell. Non-viral techniques include precipitation of DNA with calcium phosphate (Chen et al., 1987; Sompayrac et al., 1981) or with polybrene (Aubin et al., 1988); direct introduction of DNA using cell electroporation (Neuman et al., 1982) or DNA microinjection (Capecchi, 1980); complexation of DNA with polycations (Kabanov et al., 1995); and DNA incorporation in reconstructed virus coats (Schreier et al., 1992; Schreier et al., 1995).

Cationic lipids have become important reagents for gene transfer in vitro and in vivo. Several clinical trials approved by the NIH are in progress (Ledley, 1994 and Ledley, 1995). In terms of transfection efficiency, virus-based vectors are superior to all other DNA transfection methods. Several different viral vectors have been developed and are in clinical trials including those derived from murine leukemia viruses (retroviruses), adeno-associated virus, and adenovirus (reviewed in Ledley, 1996).

There have been only a few reports of nucleic acids entering mitochondria, and most have focused on the nuclear encoded RNA component of the mitochondrial RNA processing activity, RNase MRP (Chang et al., 1987; Li et al., 1994). The uptake of exogenous DNA into mitochondria involving the protein import pathway has been reported from two laboratories. Vestweber et al. (1989) achieved uptake of a 24-bp both single- and double-stranded oligonucleotide into yeast mitochondria by coupling the 5′ end of the oligonucleotide to a precursor protein consisting of the yeast cytochrome c oxidase subunit IV presequence fused to a modified mouse dihydrofolate reductase. More recently, Seibel et al. (1995) reported the import into the mitochondrial matrix of double-stranded DNA molecules conjugated to the amino-terminal leader peptide of the rat ornithine-transcarbamylase. Both studies, however, were done with isolated mitochondria, not addressing the question of how oligonucleotide-peptide conjugates will pass the cytosolic membrane and reach mitochondrial proximity. Negatively-charged biological cell surfaces and lysosomal degradation establish major obstacles, which are very unlikely to be overcome by single oligonucleotide-peptide complexes.

The need for mitochondria-specific delivery systems arises from the central role mitochondria play in a multitude of metabolic pathways (Weissig, 2005; Weissig et al., 2004; Murphy et al., 2000). Despite the progress being made in developing delivery systems for DNA and bioactive molecules into intact cells and into nuclei of intact cells, there is a need for an efficient method for introducing DNA and bioactive molecules into mitochondria of intact cells.

### BRIEF SUMMARY OF THE INVENTION

This invention is directed to mitochondriotropic phospholipid vesicles, i.e., mitochondriotropic liposomes, that comprise a hydrophobized amphiphilic delocalized cation, such as those comprising, e.g., a triphenylphosphonium or a quinolinium moiety, incorporated into the phospholipid membrane of the vesicles, or liposomes. The hydrophobized portion of the amphiphilic delocalized cation, e.g., a fatty acid or other phospholipid derivative, is embedded in the phospholipid membrane of the liposome, and the amphiphilic portion of the cation is exposed on the surface of the liposome.

Mitochondriotropic liposomes according to the invention constitute a mitochondria-targeted drug delivery system, which permits the transport of a high payload of therapeutic water-soluble molecules in their native (i.e., active) state specifically and exclusively to mitochondria in living mammalian cells. This invention combines into one delivery system the mitochondria-specificity of amphiphilic delocalized cations with the ability of liposomes to encapsulate a large variety of water-soluble molecules.

Thus, the compositions according to the invention provide a universally applicable drug delivery system, which delivers in vitro and in vivo highly water-soluble molecules (such as low-molecular weight drugs, peptides, peptide-nucleic acids, saccharides and oligonucleotides) selectively and exclusively to mitochondria in living mammalian cells, including malignant transformed cells.

The delivery of both small drug molecules and large macromolecules to and into mitochondria provides the basis for a large variety of cytoprotective and cytotoxic therapies. For example, the delivery of therapeutic DNA and RNA such as antisense oligonucleotides, ribozymes, plasmid DNA expressing mitochondrial encoded genes as well as wild-type mtDNA can provide the basis for treatment of mitochondrial DNA diseases. The delivery of antioxidants can protect mitochondria from oxidative stress caused by a variety of insults, perhaps even contributing to slowing down the natural aging process. The delivery of mitochondria-specific naturally occurring toxins or synthetic drugs such as photosensitizers can open up avenues for new anticancer therapies. Moreover, delivering molecules known to trigger apoptosis by directly acting on mitochondria can overcome the apoptosis-resistance of many cancer cells. The delivery of drugs targeting mitochondrial uncoupling proteins can become a basis for treating obesity, and the delivery of peptides and proteins can become the basis for the treatment of a huge variety of other mitochondrial disorders.

### BRIEF DESCRIPTION OF THE DRAWINGS

Other features and advantages of the invention will be apparent from the following description of the preferred embodiments thereof and from the claims, taken in conjunction with the accompanying drawings, in which:

FIGS. 1A-1C show the chemical structures of typical commonly used mitochondriotropic molecules;

FIGS. 2A-2C are a schematic depiction of a method of preparing compositions according to the invention. (A) a hydrophobized mitochondriotropic triphenylphosphonium cation, STPP. (B) the cation of (A) anchored in a liposomal phospholipid bilayer membrane via an alky residue (not drawn to molecular scale). (C) a mitochondriotropic phospholipid vesicle (liposome) according to the invention;

FIG. 3 shows the ³¹P-NMR spectrum of STPP;

FIG. 4 shows the ³¹P-NMR spectrum of a liposome composition according to the invention with 20 mol % incorporated STPP. Peak A: δP (phosphate); Peak B: δP (phosphonium);

FIG. 5 shows the size distribution of mitochondriotropic liposomes according to the invention with 20 mole incorporated STPP (mean diameter: 132.1±59.6 nm);

FIG. 6 is a graph showing the zeta potential of mitochondriotropic liposomes according to the invention with various amounts of incorporated STPP;

FIGS. 7A and 7B show epifluorescence microscopy images of BT20 cells: A: Cells incubated with Rhodamine-PE labeled STPP liposomes. B: Mitochondria in BT20 cells stained with MitoTracker Red;

FIG. 8 represents a micrograph of DNA gel separation analysis of COLO205 cells incubated with C6 ceramide-loaded mitochondriotropic liposomes according to the invention for 18 hrs, followed by extraction of their DNA. The concentration of ceramide in all samples was 25 μM. Lane A—DNA marker; lane B—untreated control; lane C—ceramide-loaded plain liposomes; lane D—ceramide loaded STPP liposomes according to the invention; lane E—free ceramide. Black arrows indicate the apoptotic DNA ladder. Total amount of DNA was identical in all lanes; and

FIG. 9 is a bar graph showing the biodistribution of PEGylated mitochondriotropic liposomes according to the invention.

### DETAILED DESCRIPTION OF THE INVENTION

The compositions of the invention provide a universally applicable drug delivery system, which delivers in vitro and in vivo highly water-soluble molecules (low-molecular weight drugs, peptides, peptide-nucleic acids, saccharides, oligonucleotides) selectively and exclusively to mitochondria in living mammalian cells including malignant transformed cells. The compositions according to the invention can be used to address the significant problems caused by damaged or diseased mitochondria. Recently, more than fourteen groups of potential mitochondrial drugs or mitochondrial drug targets have been summarized (Weissig et al., 2004). The vast majority of the recently published reviews center around drug targets related to the crucial role mitochondria play during apoptosis (Morisaki et al., 2003; Kotake et al., 2003; Kroemer, 2003; Malisan et al., 2003; Galiegue et al., 2003; O'Neil et al., 2003; Pastorino et al., 2003; Granville et al., 2003; Halestrap et al., 2003; Waldmeier et al., 2003; Crompton, 2003; Sordet et al., 2003). Another area of high interest is the protection of mitochondria from oxidative stress (James et al., 2004; Green et al., 2004; Coulter et al., 2000). An additional area of interest is disease caused by mutated mitochondrial DNA (Dimauro et al., 2001; Wallace, 1992; Wallace, 2001; Pulkes et al., 2001; Manfredi et al., 2000; D'Souza et al., 2004). A large number of treatments have been proposed in this area.

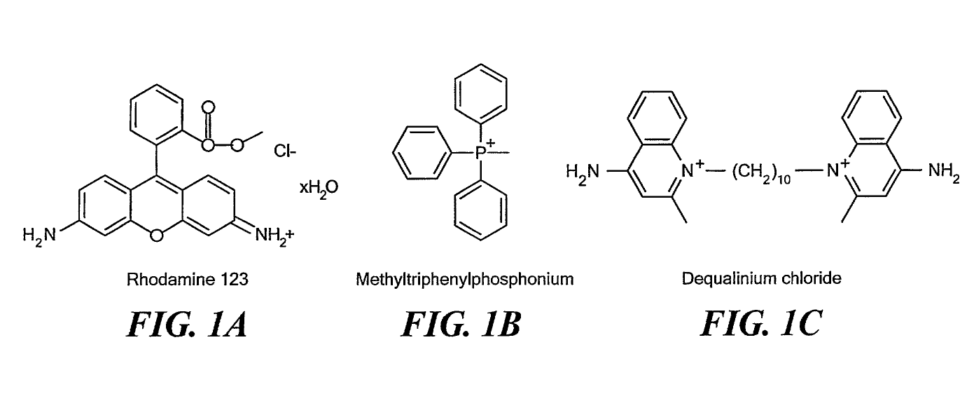

Certain amphiphilic compounds (“mitochondriotropics”) are able to cross both mitochondrial membranes leading to their accumulation in the mitochondrial matrix. FIG. 1 shows the chemical structure of representative mitochondriotropic molecules, Rhodamine 123 (compound A) has been used extensively as a stain for mitochondria in living cells since its introduction in 1982 (Chen et al., 1982). By 1969, methyltriphenylphosphonium salts (compound B) had been demonstrated to be taken up rapidly by mitochondria in living cells (Lieberman et al., 1969), and the mitochondrial accumulation of dequalinium chloride (compound C) was established during the 1980s (Weiss et al., 1987). Other examples of mitochondriotropic cations (structures not shown) are cyanine dyes such as N,N′-bis(2-ethyl-1,3-dioxolane) kryptocyanine (Oseroff et al., 1986) and Victoria Blue BO (Morgan et al., 1998).

Mitochondriotropic molecules have two structural features in common. First, they are all amphiphilic; that is, they combine a hydrophilic charged center with a hydrophobic core. Second, in all structures the n-electron charge density extends over at least three atoms or more instead of being limited to the internuclear region between the heteroatom and the adjacent carbon atom. This causes a distribution of the positive charge density between two or more atoms; that is, the positive charge is delocalized, thus the terminology “delocalized cations.” Both structural features are widely believed to be crucial for the accumulation of these organic cations inside the matrix of mitochondria. Sufficient lipophilicity combined with delocalization of the positive charge on these cations, to reduce the free energy change when the cations move from an aqueous to a hydrophobic environment, are thought to be prerequisites for their mitochondrial accumulation in response to the mitochondrial membrane potential (Weiss et al., 1987).

The compositions and methods of the invention make use of these properties of mitochondriotropic molecules by combining the hydrophobized versions of these organic cations with the ideal vesicular delivery system, the liposome. Colloidal vesicles composed of phospholipids, or liposomes, are one of the most versatile and most extensively studied drug delivery systems. Liposomes can encapsulate an unlimited variety of hydrophilic, amphiphilic and hydrophobic small molecules either in their aqueous inner space or in their lipid bilayer membranes. They are essentially nontoxic, non-immunogenic and biodegradable; that is, liposomes meet all prerequisites for an ideal drug delivery system. The surface modification of liposomes with polyethylene glycol leads to prolonged circulation times in the bloodstream (Klibanov et al., 1990), which in turn is the basis for a variety of liposome-based drugs that have been approved by the FDA and FDA-like agencies in Europe and Asia over the last decade.

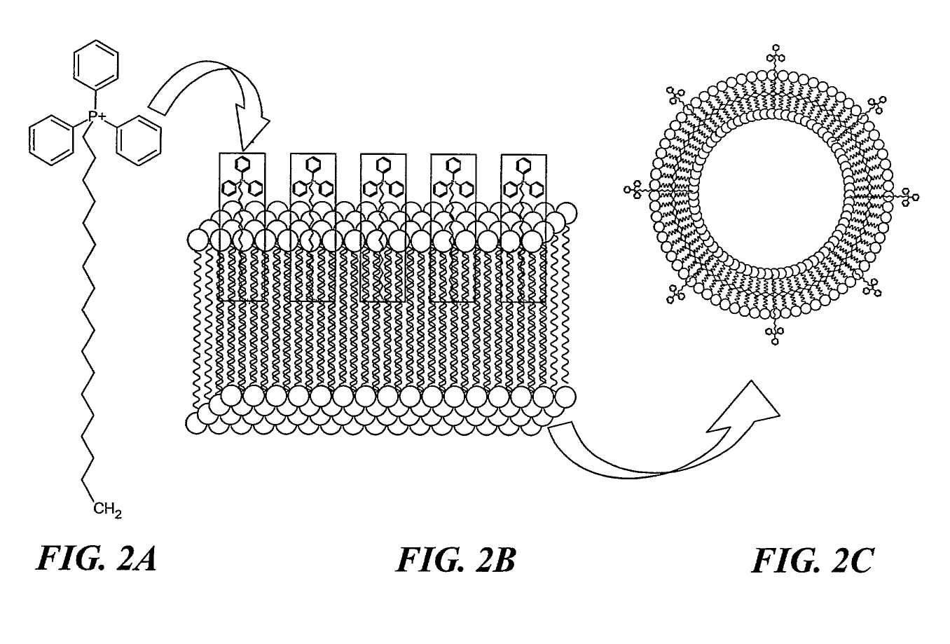

To utilize the superior drug carrier properties of liposomes for mitochondria-targeted delivery of bioactive molecules, amphiphilic delocalized cations have been hydrophobized according to the invention to form liposomes having surface-linked mitochondriotropic residues. In an exemplary embodiment according to the invention, stearyltriphenylphosphonium bromide (STPP) was synthesised by replacing the methyl group in methyltriphenylphosphonium bromide (FIG. 1, compound B) with a stearyl residue to form a hydrophobized amphiphilic delocalized cation. The hydrophobic fatty acid (stearyl) residue “anchors” the mitochondriotropic triphenylphosphonium cation in the phospholipid bilayer membrane (i.e., “attaches” it covalently to the liposomal surface), as shown schematically in FIGS. 2B and 2C.

Any therapeutic compound, e.g., a therapeutic compound previously delivered by another delivery system, is a candidate for delivery to a patient by the system of the invention. Cyclosporin A (CsA), for example, has been shown to bind with nanomolar affinity to mitochondrial cyclophilin D, which potentially makes it an interesting anti-ischaemic drug candidate (Woodfield et al., 1998). However, CsA also targets at least eight other cyclophilins inside the cell, which are likely to bind a large portion of the administered drug. Therefore, the mitochondrial concentration of CsA is difficult to predict, and an effective CsA treatment may require high, even toxic concentrations to reach the mitochondrial target (Waldmeier et al., 2003). Consequently, CsA as a potential anti-ischaemic drug would benefit from the mitochondria-specific drug carrier system according to the invention, which would be able to increase its therapeutic index.

Paclitaxel is a potent antitubulin agent used in the treatment of malignancies (Eisenhauer et al., 1998). It has recently been demonstrated that clinically relevant concentrations of paclitaxel target mitochondria directly and trigger apoptosis by inducing cytochrome c (cyt c) release in a permeability transition pore (PTP)-dependent manner (Andre et al., 2002). Unfortunately, paclitaxel has a very narrow therapeutic window (Seligson et al., 2001, which most likely reflects the existence of several drug targets inside the cell, thus making only a subset of the drug available for mitochondria (Andre et al., 2002). Consequently, paclitaxel as an anticancer drug should greatly benefit from the organelle-specific delivery system according to the invention.

Ceramide is a major sphingolipid with sphingosine as its basic structure. A lipid-signaling molecule, it helps to regulate the differentiation, proliferation, and death of cells. In the cell, ceramide is degraded with ceramidase to sphingosine, which is then phosphorylated by sphingosine kinase to form sphingosine-1-phosphate. Phosphorylated sphingosine is able to form special channels in the outer mitochondrial membrane, releasing into the cell factors that trigger apoptosis and, thus, inhibiting cancer cell growth. The organelle-specific delivery system according to the invention should significantly increase the specificity, and thus the activity, of this useful therapeutic compound.

The therapeutic compositions of the invention may be administered orally, topically, or parenterally (e.g., intranasally, subcutaneously, intramuscularly, intravenously, or intra-arterially) by routine methods in pharmaceutically acceptable inert carrier substances and solutions. For example, the compositions of the invention may be administered in a sustained release formulation using a biodegradable biocompatible polymer. Filled mitochondriotropic phospholipid vesicles according to the invention can be administered in a dosage of 0.25 μg/kg/day to 5 mg/kg/day, and preferably 1 μg/kg/day to 500 μg/kg/day. Optimal dosage and modes of administration can readily be determined by conventional protocols.

The therapeutic compositions may be administered either systemically or in a site-specific manner. Additional targeting agents may be added to the surface of the mitochondriotropic liposomes of the invention, where appropriate, for targeted delivery to a specific cell type. In the case of treatment for cancer, carcinoma cells are known to have a higher membrane potential than normal cells, which results in thier preferential uptake of the mitochondriotropic liposomes of the invention.

The therapeutic compositions of the invention can be administered independently or co-administered with another active agent. It is contemplated that the therapeutic compositions of the invention will be particularly useful as antineoplastic agents when co-administered with, e.g., cis-Platin or methotrexate.

The following examples are presented to illustrate the advantages of the present invention and to assist one of ordinary skill in making and using the same. These examples are not intended in any way otherwise to limit the scope of the disclosure.

### EXAMPLE I

### Synthesis and Characterization of the Bromide Salt Containing Stearyl-Triphenylphosphonium Cation as a Hydrophobized Amphiphilic Delocalized Cation According to the Invention

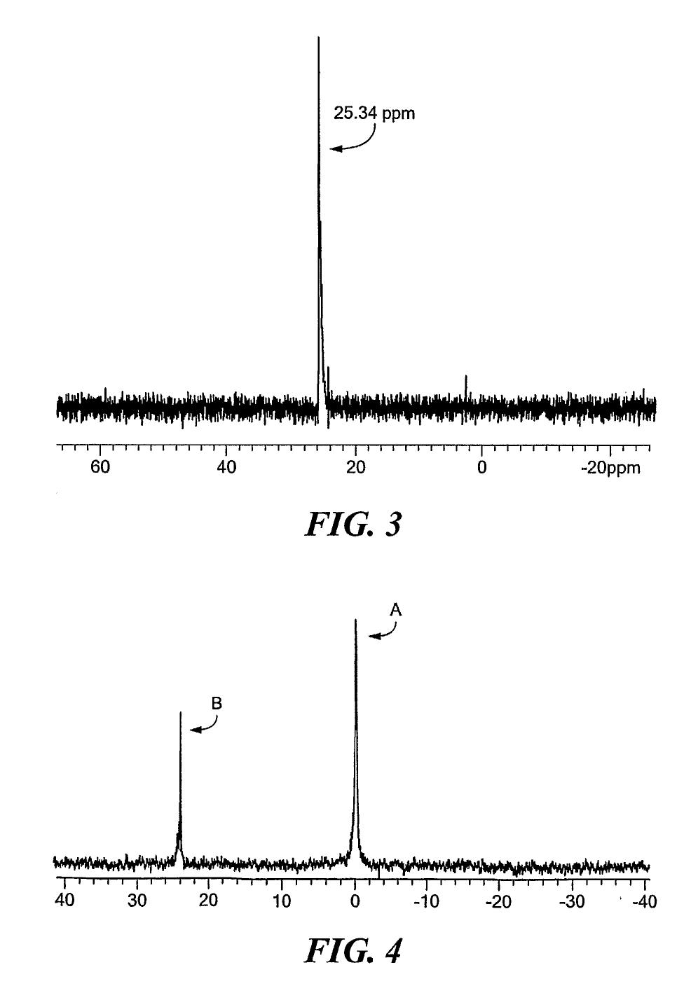

Stearyl-triphenylphosphonium bromide (according to FIG. 2A) was synthesized by heating stearyl bromide and triphenylphosphine (FIG. 1B) under reflux in xylene according to a protocol for the synthesis of analogous tertiary phosphonium salts (see, Materials and Methods). Isolation by column chromatography on silica gel and recrystallization from diethylether yielded a chromatographically pure product, which was identified by ¹H-NMR as described in Materials and Methods. FIG. 3 shows the ³¹P-NMR spectrum of STPP. The observed ³¹P chemical shift of STPP is at 25.34 ppm, well within the range of 20.9-26.2 ppm as described for a series of alkyl- and aryl triphenylphosphonium salts (Kiddle, 2000). In comparison, the ³¹P chemical shift of triphenylphosphine was found to be −4.48 ppm, which is in perfect agreement with the published ³¹P-NMR data (Allen and Taylor, 1982).

### EXAMPLE II

### Preparation and Characterization of Liposomes According to the Invention with Surface-Linked Triphenylphosphonium Cations

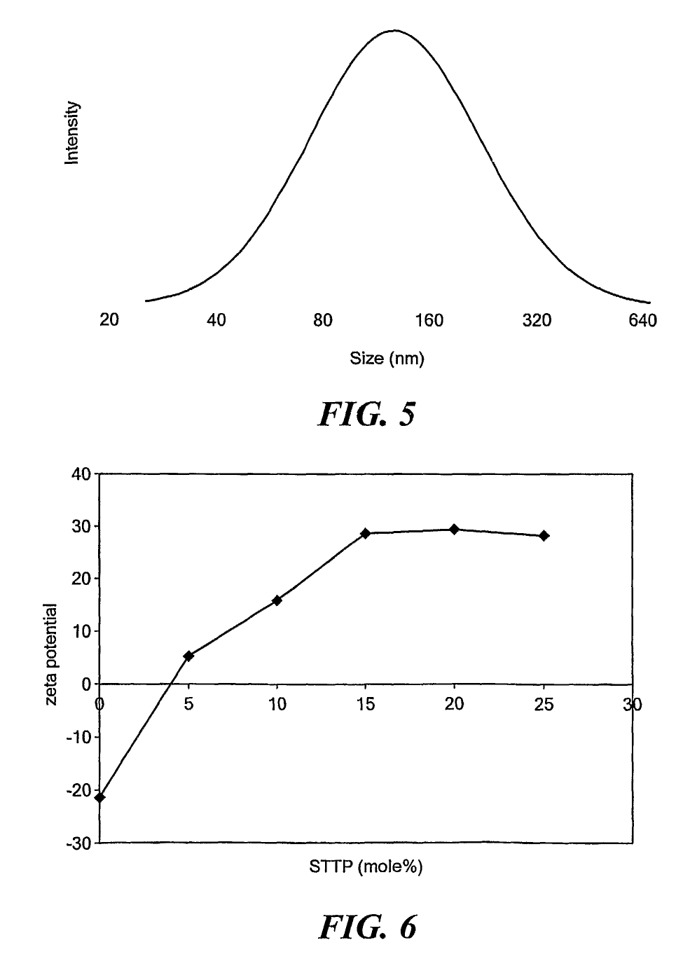

The preparation of liposomes in the presence of hydrophilic molecules that have been hydrophobized via linkage to fatty acid or phospholipid derivatives results in the covalent “anchoring” of the hydrophilic moiety to the liposomal surface. Liposomes according to the invention were prepared in the presence of STPP according to standard protocols (Lasch et al., 2003). FIG. 2B shows schematically the alkyl (i.e., stearyl) residue mediated “anchoring” of the triphenylphosphonium cation in the liposomal phospholipid bilayer membrane. STPP liposomes, i.e., liposomes with surface-linked triphenylphosphonium cations, were isolated using a Sephadex G-15 column and characterized by ³¹P-NMR (FIG. 4), size distribution analysis (FIG. 5) and zeta potential measurements (FIG. 6).

As expected (see, FIG. 4), the ³¹P-NMR spectrum of STPP-liposomes shows two chemical shifts correlating to the phosphorus in the phosphate groups of the lipid (Peak A) and to the positively charged phosphorus of STPP (Peak B). No differences in both chemical shifts between the free compounds (i.e., free STPP and free phospholipid) and the liposomal incorporated molecules could be found, indicating that the triphenylphosphonium group of STPP does not seem to interact with the bilayer membrane. This in turn supports the conclusion that STPP is anchored in the lipid membrane as schematically shown in FIG. 3. The size of liposomes with 20 mol % incorporated STPP (FIG. 5) is 132.1±59.6 nm, slightly larger than would have been expected for liposomes prepared by probe sonication, an effect that is most-likely due to the presence of the STPP. Liposome size did not change significantly upon storage at 4° C. over several days. The zeta-potential of STPP-liposomes (FIG. 6) seems to increase linearly with increasing amounts of incorporated STPP until it reaches a plateau between 15 and 20 mol % STPP. Whether this observed plateau is due to a limitation of the maximal amount of STPP incorporable in liposomes has not been investigated, but subsequently, for all in vitro studies, liposomes with 20 mol % STPP were used.

### EXAMPLE III

### Intracellular Distribution of STPP Liposomes

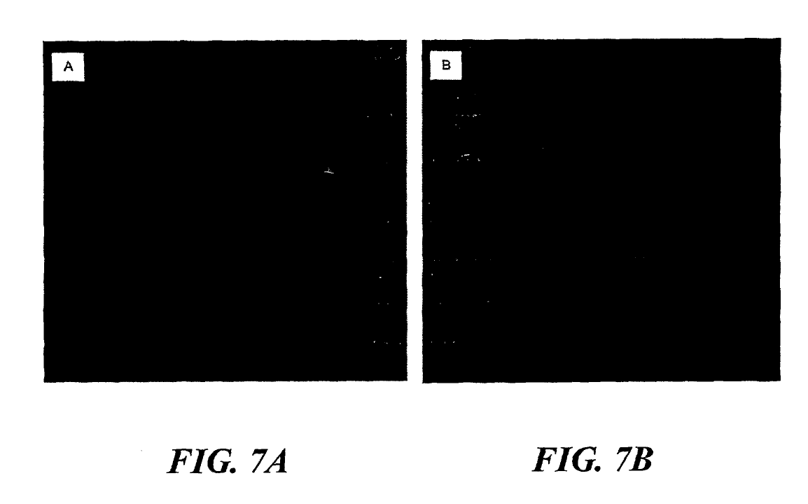

To study the cellular uptake and intracellular distribution of STPP liposomes, cells of the breast cancer cell line BT 20 were incubated with fluorescence-labeled STPP liposomes for 1 h in serum-free medium. To remove non-internalized liposomes, cells were thoroughly washed and allowed to grow for another hour in complete medium. Typically obtained epifluorescence microscopic images are shown in FIGS. 7A and 7B. FIG. 7A displays cells incubated with STPP liposomes that have been labeled by incorporation of 0.5 mol % Rhodamine-PE, while FIG. 7B shows cells, the mitochondria of which have been specifically stained with Mitotracker red. On comparing FIG. 7A with FIG. 7B, it can be seen that cells incubated with STPP liposomes display the same distinct fluorescence pattern as cells stained with the mitochondria-specific dye. Such a comparison of staining patterns has been used by Filipovska et al. (2004) to reveal the localization of labeled thiol proteins inside mitochondria and by Geromel et al. (2001) to show the localization of labeled oligonucleotides at and inside mitochondria within living mammalian cells. Based on the fact that the fluorophore in our STPP liposomes was covalently linked to phospholipids and not to the mitochondriotropic entity, i.e., to STPP, it can be concluded from FIGS. 7A and 7B that at least partially intact phospholipid vesicles have accumulated at or near the site of mitochondria.

### EXAMPLE IV

### Ceramide Encapsulated into Mitochondriotropic Liposomes According to the Invention Displays Increased Apoptotic Activity

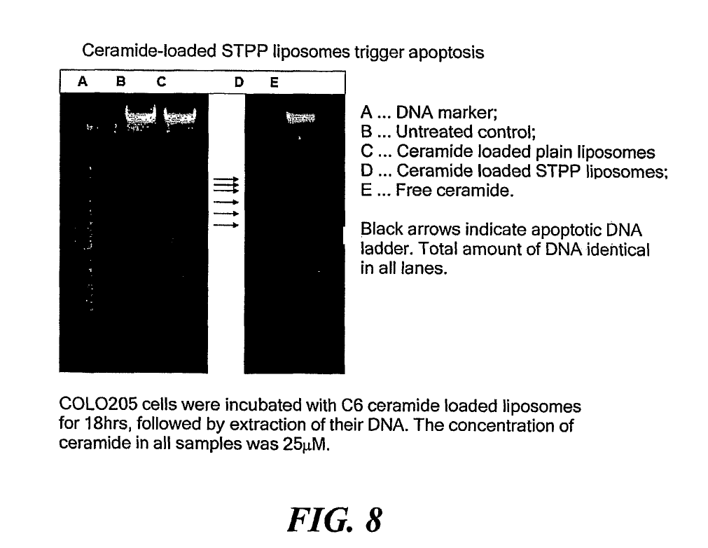

Results of a preliminary study suggest that the pro-apoptotic activity of C6 ceramide is increased when formulated in mitochondriotropic, i.e., STPP, liposomes according to the invention. FIG. 8 represents a micrograph of DNA gel separation analysis of cells of the colon cancer cell line COLO205 incubated with C6 ceramide-loaded mitochondriotropic liposomes according to the invention for 18 hrs, followed by extraction of their DNA. The concentration of ceramide in all samples was 25 μM. Lane A—DNA marker; lane B—untreated control; lane C—ceramide-loaded plain liposomes; lane D—ceramide loaded STPP liposomes according to the invention; lane E—free ceramide. Black arrows indicate the apoptotic DNA ladder. The total amount of DNA was identical in all lanes.

At identical ceramide concentrations, neither cells treated with ceramide loaded plain liposomes (Lane C, FIG. 8) nor cells treated with free ceramide (Lane E, FIG. 8) showed the DNA laddering typical of apoptosis. Cells treated with ceramide encapsulated in STPP liposomes, however, do not seem to have any intact DNA anymore (empty well), but do display a DNA ladder (degraded DNA) as indicated by the black arrows (Lane D, FIG. 8).

### EXAMPLE V

### Biodistribution of PEGylated Mitochondriotropic Liposomes

Nude mice were inoculated subcutaneously into the left flank with COLO205 colon cancer cells. After the tumor size had reached 2-3 mm in diameter, 0.1 ml samples of radio-labeled liposomes (5 μCi total) were injected into the tail vein and the animals were sacrificed after 24 hours. Liposomes carrying polyethylene glycol (3′ PEG5000) had a zeta potential of −10.6, whereas liposomes loaded with 100 STPP as well as with 3% PEG5000 exhibited a zeta potential of +12.1.

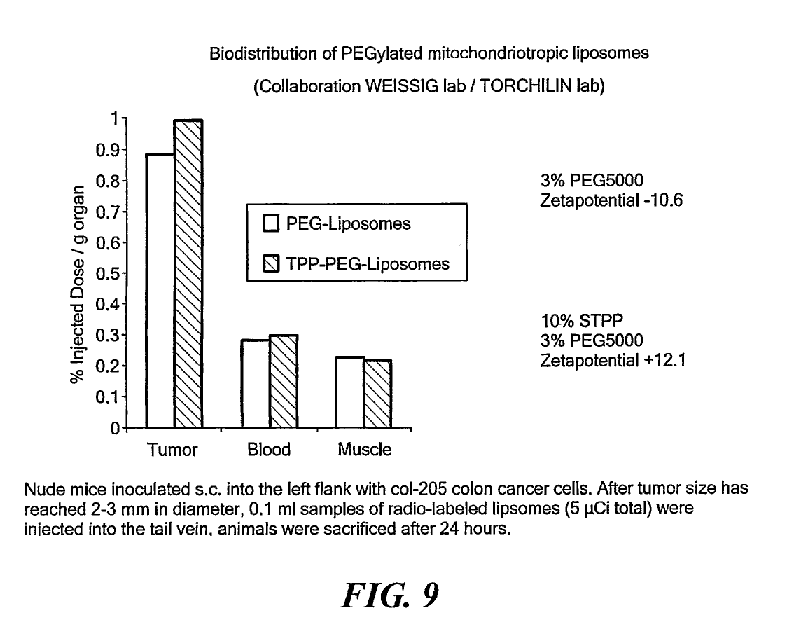

Referring to FIG. 9, it can be seen that adding a mitochondriotropic cation to the surface of PEGylated liposomes does not change the biodistribution pattern of the liposomes in sites as varied as tumor, blood or muscle. Thus, the delivery system of the invention is completely blood compatible.

Materials and Methods

Materials

Triphenylphosphine (TPP), stearyl bromide, and organic solvents were purchased from Fisher Scientific and used without further purification. Egg phosphatidylcholine (PC) and cholesterol (Ch) were obtained from Avanti Polar Lipids. The CellTiter AQueous One Solution Cell Proliferation Assay kit was purchased from Promega, and all fluorescence dyes used in this study were obtained from Molecular Probes.

Synthesis of Stearyltriphenylphosphonium Bromide (STPP)

Following a protocol for the synthesis of analogue tertiary phosphonium salts (Rideout et al., 1989), stearyl bromide (1.93 g, 5.5 mmol) and triphenylphosphine (1.52 g, 5.8 mmol) were heated under reflux for 20 h in freshly distilled anhydrous xylene (30 mL). The progress of the reaction was monitored by thin layer chromatography (TLC) on silica gel plates (Silica Gel 60, F-254) using iodine as detection reagent. The solvent was removed on a rotary evaporator to obtain crude yellowish oil. The crude was purified by silica gel column chromatography (75 g) using methanol:chloroform (5:95) as an eluent. Purified STPP, obtained as a colorless oil, crystallized on standing and was recrystallized from ether to yield pure STPP in 35-45% yields. The product was characterized by ¹H-NMR (CDCl₃) and ³¹P-NMR (with external 85% H₃PO₄ as reference) on a VARIAN Mercury 300 NMR spectrometer: ¹H NMR: 7.68-7.88 (m, 15H), 3.7-3.8 (m, 2H), 1.55-1.65 (t, 4H), 1.2-1.35 (m, 28H), 0.7-0.8 (t, 3H); ³¹P-NMR: 25.34.

Preparation of Liposomes with Incorporated STPP (STPP-Liposomes)

STPP-liposomes were prepared by probe sonication according to the general method of liposome preparation described in Lasch et al. (2003). Briefly, a mixture of lecithin, cholesterol, hydrophobized polyethylene glycol and STPP (PC/Ch/hPEG/STPP=62/15/3/20, molar ratio; final total lipid 25 mg/mL) was dissolved in chloroform followed by removal of the organic solvent using a rotary evaporator. After the addition of 5 mM HEPES (pH 7.4) to the dry lipid film, the sample was probe sonicated with a Sonic Dismembrator (Model 100, Fischer Scientific) at a power output of approximately 10 watts for 30 min. To remove any titanium particles shed from the tip of the probe during sonication, the sample was centrifuged for 10 min at 3000×g. The formed STPP-liposomes were separated from free, i.e., non-incorporated STPP, by gel filtration chromatography on a Sephadex G-15 column. The liposomal incorporation of STPP was confirmed by ³¹P-NMR spectroscopy.

Size Distribution Analysis

The liposome size and size distribution were determined by quasielastic laser light scattering using a Coulter N4 Submicron Particle Size Analyzer.

Zeta Potential Measurements of STPP-Liposomes

The zeta potential of STPP liposomes was determined at 2.5 V, 657 nm, 2.00 Hz and 25° C. using the Zeta Potential Analyzer Version 3.26 from Brookhaven Instruments Corporation. For each measurement, 10 μl liposome solution (total lipid, 25 mg/ml; STPP content varying between 0 and 25 mol %) were added into 2 mL HBS, pH 7.4 and incubated until temperature equilibration was attained.

Intracellular Distribution of STPP Liposomes

For analyzing their intracellular distribution pattern by fluorescence microscopy, STPP liposomes (20 mol % STPP) were labeled with 0.5 mol % Rhodamine-PE (Aex=550 nm, Aem=590 nm). BT-20 cells were grown on 22 mm cover slips in 6-well plates to about 85% confluence. After the medium had been replaced with serum-free medium, 35 μl STPP liposomes (25 mg/mL lipid) were added to each well. Following a 1 h incubation, the medium containing non-internalized liposomes was removed, cells were thoroughly washed, allowed to grow for another hour in complete medium and analyzed by epifluorescence microscopy using a BX61 Olympus model. For visualizing mitochondria in cells not treated with STPP liposomes, cells were co-incubated for 5-7 min with Mitotracker Red CMXRos. Images were taken with an Olympus camera on an Olympus BX61 microscope using a TRITC filter for Mitotracker and for Rhodamine. Photographs were taken digitally and processed using Adobe Photoshop 6.0.

### REFERENCES

Allen, D. W., Taylor, B. F. (1982). The chemistry of heteroarylphosphorus compounds: part 15. Phosphorus-31 nuclear magnetic resonance studies of the donor properties of hetero-arylphosphines towards selenium and platinum (II). J. Chem. Soc. Dalton Trans, 5154.
Andre, N., Carre, M., Brasseur, G. (2002). Oaclitaxel targets mitochondria upstream of caspase activation in intact human neuroblastoma cells. FEBS Lett. 532:256-260.
Boddapati, S. v., Tongcharoensirikul, P., Hanson, R. N., D'Souza, G., Torchilin, V. P., Weissig, V., Mitochondriotropic Liposomes, J Liposome Research 15 (2005) 49-58.
Brown, G. c., Nicholls, D. G., Cooper, C. E. (1999). Mitochondria and cell death. In: Mitochondria and Cell Death. Princeton, N.J.: Princeton University Press.
Bums, R. J., Smith, R. A. J., Murphy, M. P. (1995). Synthesis and characterization of thio butyltriphenylphosphonium bromide, a novel thiol reagent targeted to the mitochondrial matrix. Arch. Biochem. Biophys. 322:60-68.
Chen, L. B., Summerhayes, I. C., Johnson, L. V., Walsh, M. L., Bernal, D. D., Lampidis, T., (1982). Probing mitochondria in living cells with rhodamine 123. Cold Spring Harb. Symp. Quant, Biol., 46:141-155.
Coulter, C. V., Kelso, G. F., Lin, T. K., Smith, R. A., Murphy, M. P. (2000). Mitochondrially targeted antioxidants and thiol reagents. Free Radic. Bioi. Med. 28(10):1547-1554.
Coulter, C. V., Smith, R. A. L., Murphy, M. P. (2001). Synthesis, characterization, and biological properties of a fullerene triphenylphosphonium salt. Fuller Sci. Technol. 9:339-350.
Crompton, M., (2003). On the involvement of mitochondrial intermembrane junctional complexes in apoptosis. Curr. Med. Chem. 10:1473-1484.

Cudd, A., Nicolau, C. (1985). Intracellular fate of liposome-encapsulated DNA in mouse liver: analysis using electron microscope autoradiography and subcellular fractionation. Biochim. Biophys. Acta 845(3):477-491.
Cudd, A., Nicolau, C. (1986). Interaction of intravenously injected liposomes with mouse liver mitochondria. A fluorescence and electron microscopy study. Biochim. Biophys. Acta 860(2):201-214.
Cudd, A., Labbe, H., Gervais, M., Nicolau, C. (1984). Liposomes injected intravenously into mice associate with liver mitochondria. Biochim. Biophys. Acta 774(2):169-180.
Dimauro S., Schon, E. A., (2001). Mitochondrial DNA mutations in human disease. Am. J. Med. Genet. 106:18-26.
D'Souza, G. G. M., Weissig, V., (2004). Approaches to mitochondrial gene therapy. Curr. Gene. Ther. 4:317-328.
Eisenhauser, E. A., Vermorken, J. B., (1998). The taxoids: comparative clinical pharmacology and therapeutic potential. Drugs 55:5-30.
Filipovska, A., Eccles, M. R., Smith, R. A. J., Murphy, M. P. (2004). Delivery of antisense peptide nucleic acids (PNAs) to the cytosol by disulphide conjugation to a lipophilic cation. FEBS Leu. 556:180-186.
Galigue, S., Tinel, N., Casellas, P., (2003). The peripheral benzodiazepine receptor: a promising therapeutic drug target. Curr. Med. Chem. 10:1563-1572.
Geromel, V., Cao, A., Briane, D., Vassy, J., Rotig, A., Rustin, P., Coudert, R., Rigaut, 1. P., Munnich, A., Taillandier, E. (2001). Mitochondria transfection by oligonucleotides containing a signal peptide and vectorized by cationic liposomes. Antisense Nucleic Acid Drug Dev. 11(3):175-180.

Granville, D. J., Gottlieb, R. A., (2003). The mitochondrial voltage-dependent anion channel (VDAC) as a therapeutic target for initiating cell death. Curr. Med. Chem. 10:1527-1533.
Green, K., Brand, M. D., Murphy, M. P., (2004). Prevention of mitochondrial oxidative damage as a therapeutic strategy in diabetes. Diabetes, 53:110-118.
Halestrap, A. P., Brennerb., C., (2003). The adenine nucleotide translocase: a central component of the mitochondrial permeability transition pore and key player in cell death. Curr. Med. Chem. 10: 1507-1525.
Huth, D., Wieschollek, A., Garini, Y., Schubert, R., Peschka-Suss, R. (2004). Fourier transformed spectral bio-imaging for studying the intracellular fate of liposomes. Cytometry 57A(1):10-21.
James, A. M., Smith, R. A., Murphy, M. P., (2004). Antioxidant and prooxidant properties of mitochondrial Coenzyme Q. Arch. Biochem. Biophys. 423:47-56.
Kiddle, J. C. (2000). Microwave irradiation in organophosphorus chemistry. Part 2: synthesis of phosphonium salts. Tetrahedron. Lett. 41:1339-1341.
Klibanov, A. L., Maruyama, K., Torchilin, V. P., Huang, L. (1990). Amphipathic polyethyleneglycols effectively prolong the circulation of liposomes. FEBS Lett. 268:235-237.
Kotake Y. Ohta S. (2003). MPP+ analogs acting on mitochondria and inducing neuro-degeneration. Curr. Med. Chem., 10:2507-2516.
Kroemer, G. (2003). The mitrochondrial permeability transition pore complex as a pharmacological target, Curr. Med. Chem., 10:1469-1472.
Larson, N. G., Luft, R. (1999). Revolution in mitochondrial medicine. FEBS Leu. 455:199 202.
Lasch, J., Weissig, V., Brandl, M. (2003). Preparation of liposomes. In: Torchilin, V. P., Weissig, V., eds. Liposomes-A Practical Approach. 2nd ed. Oxford: Oxford University Press. pp. 3-30.
Liberman, E. A., Topaly, V. P., Tsofina, L. M., Jasaitis, A. A., Skulachev, V. P. (1969). Mechanism of coupling of oxidative phosphorylation and the membrane potential of mitochondria. Nature, 222:1076-1078.
Malich, G., Markovic, B., Winder, C. (1997). The sensitivity and specificity of the MTS tetrazolium assay for detecting the in vitro cytotoxicity of 20 chemicals using human cell lines. Toxicology 124(3):179-192.
Malisan, F., Testi, R. (2003). Mitochondrial lipids as apoptosis regulators, Curr. Med. Chem., 10:1573-1580.
Manfredi, G., Beal, M. F., (2000). The role of mitochondria in the pathogenesis of neurodegenerative diseases. Brain Pathol. 10:462-472.
Morgan, J., Whitaker, J. E., Oseroff, A. R. (1998). GRP78 induction by calcium ionophore potentiates photodynamic therapy using the mitochondrial targeting dye Victoria blue BO. Photochem. Photobiol. 67:115-164.
Morisaki T, Katano M. (2003). Mitochondria-targeting therapeutic strategies for overcoming chemotesistance and progression of cancer. Curr. Med. Chem. 10:2517-2521.
Murphy, M. P., Smith, R. A. (2000). Drug delivery to mitochondria: the key to mitochondrial medicine. Adv. Drug. Deliv. Rev. 41(2):235-250.
Niedermann, G., Weissig, V., Sternberg, B., Lasch, J. (1991). Carboxy acyl derivatives of cardiolipin as four-tailed hydrophobic anchors for the covalent coupling of hydrophilic proteins to liposomes. Biochim. Biophys. Acta 1070(2):401-408.
O'Neil, J. W., Hockenbery, D. M., (2003). Bcl-2-related proteins as drug targets. Curr. Med. Chem., 10:1553-1562.
Oseroff, A. R., Ohauoa, D., Ara, G., McAuliffe, D., Foley, J., Cincotta, L., (1986). Intramitochonrial dyes allow selective in vitro photolysis of carcinoma cells. Proc. Natl. Acad. Sci. 83:9729-9733.
Pastorino, J. G., Hoek, J. B., (2003). Hexokinase II: the integration of energy metabolism and control of apoptosis. Curr. Med. Chem., 10:1535-1551.
Pulkes, T., Hanna, H. G., (2001). Human mitochondrial DNA diseases. Adv. Drug Deliv. Rev. 49:27-43.
Rideout, D. c., Calogeropoulou, T., Jaworski, J. S., Dagnino, R. Jr., McCarthy, M. R. (1989). Phosphonium salts exhibiting selective anti-carcinoma activity in vitro. Anti-cancer Drug Des. 4(4):265-280.
Schon, E. A., DiMauro, S. (2003). Medicinal and genetic approaches to the treatment of mitochondrial disease. Curro Med. Chem. 10:1241-1253. Singh, K. K. (2000). Mitochondrial me and the mitochondrion journal. Mitochondrion 1(1):1-2.
Seligson, A. L., Terry, P. C., Bressi, J. C., Douglass, J. G., Sovak, M. (2001). A new prodrug of paclitaxel: synthesis of Protaxel. Anti-Cancer Drugs, 12:305-313.
Smith, R. A., Porteous, C. M., Coulter, C. V., Murphy, M. P. (1999). Selective targeting of an antioxidant to mitochondria. Eur. J. Biochem. 263(3):709-716.
Smith, R. A., Porteous, C. M., Gane, A. M., Murphy, M. P. (2003). Delivery of bioactive molecules to mitochondria in vivo. Proc. Natl. Acad. Sci. U.S.A. 100(9):5407-5412.
Sordet, O., Khan, Q. A., Khon, K. W., Pommier, Y., (2003). Apoptosis induced by toposiomerase inhibitors. Curr. Med. Chem. Anti-Cancer Agents, 3:271-290.
Straubinger, R. M., Papahadjopoulos, D., Hong, K. L. (1990). Endocytosis and intracellular fate of liposomes using pyranine as a probe. Biochemistry 29(20):4929-4939.
Torchilin, V. P., Weissig, V., Martin, F. J., Heath, T. D., New, R. R. C. (2003). Surface modification of liposomes. In: Torchilin, V. P., Weissig, V., eds. Liposomes-A Practical Approach. 2nd ed. Oxford: Oxford University Press. pp. 193-230.
Waldmeier, P. C., Zimmermann, K., Quian, T., Tintelnot-Blomlery, M., Lemaser, J. J., (2003). Cyclophilin D as a drug target. Curr. Med. Chem. 10:1485-1506.
Wallace, D. C., (1992). Diseases of the mitochondrial DNA. Ann. Rev. Biochem. 61:1175-1212.
Wallace, D. C., (2001). A mitochondrial paradigm for degenerative diseases and ageing. Novartis Found. Symp. 235:247-263.
Weissig, V. (2003). Mitochondrial-targeted drug and DNA delivery. Crit. Rev. Ther. Drug Carr. Syst. 20(1):1-62.
Weissig, V. (2005). Targeted drug delivery to mammalian mitochondria in living cells. Expert Opin. Drug Deliv. 2(1):89-102.
Weissig, V., Boddapati, S. V., D'Souza, G. G. M., Cheng, S. M. (2004). Targeting of low-molecular weight drugs to mammalian mitochondria. Drug Des. Rev. (Online) 1:15-28.
Weissig, V., Cheng, S. M., D'Souza, G. (2004). Mitochondrial pharmaceutics. Mitochondrion 3:229-244.
Weissig, V., Gregoriadis, G. (1993). Coupling of aminogroup-bearing ligands to liposomes. In: Gregoriadis, G., ed, Liposome Technology. Boca Raton: CRC Press. 231-248.
Weissig, V., Lasch, J., Gregoriadis, G. (1989). Covalent coupling of sugars to liposomes. Biochim. Biophys. Acta 1003(1):54-57.
Weissig, V., Lasch, J., Klibanov, A. L., Torchilin, V. P. (1986). A new hydrophobic anchor for the attachment of proteins to liposomal membranes. FEBS Lett. 202(1):86-90.
Weiss, M. J., Wong, J. R., Ha, C. S., Bleday, R., Salem, R. R., Steele, G. D. Jr., Chen, L. B. (1987). Dequalinium, a topical antimicrobial. agent, displays anticarcinoma activity based on selective mitochondrial accumulation. Proc. Natl. Acad. Sci. U.S. A. 84(15):5444-5448.
Woodfield, K., Ruck, A., Bridiczka, D., Halestrap, A. P., (1998) Direct demonstration of a specific interaction between cyclophilin D and the adenine nucleotide translocase confirms their role in the mitrochondrial permeability transition. Biochem. J., 336:287-290.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A mitochondriotropic phospholipid vesicle comprising
a liposome having a phospholipid membrane, a surface portion and a hydrophilic interior portion; and
a hydrophobized amphiphilic delocalized cation incorporated into the phospholipid membrane of said liposome.

2. The vesicle of claim 1, wherein said hydrophobized amphiphilic delocalized cation is incorporated covalently into said phospholipid membrane.

3. The vesicle of claim 1, wherein said hydrophobized amphiphilic delocalized cation is incorporated non-covalently into said phospholipid membrane.

4. The vesicle of claim 1, further comprising a bioactive agent incorporated into said liposome.

5. The vesicle of claim 4, wherein said bioactive agent is water-soluble and is incorporated into said hydrophilic interior portion.

6. The vesicle of claim 5, wherein said water-soluble bioactive agent is selected from the group consisting of nucleic acids, oligonucleotides, antioxidants, toxins, proteins and peptides.

7. The vesicle of claim 4, wherein said bioactive agent is incorporated into said phospholipid membrane.

8. The vesicle of claim 1, wherein said hydrophobized amphiphilic delocalized cation comprises a triphenylphosphonium moiety.

9. The vesicle of claim 1, wherein said hydrophobized amphiphilic delocalized cation comprises a quinolinium moiety.
